(19) United States
(12) Patent Application Publication
(10) Pub. No.: US 2026/0174419 A1
(43) Pub. Date: Jun. 25, 2026

(54) DEVICE AND METHOD FOR MITIGATING FEMALE URINARY AND FECAL INCONTINENCE

(71) Applicant: Claudine Hélène Joly-King, Winston-Salem, NC (US)

(72) Inventor: Claudine Hélène Joly-King, Winston-Salem, NC (US)

(21) Appl. No.: 19/421,805

(22) Filed: Dec. 16, 2025

Related U.S. Application Data
(60) Provisional application No. 63/736,263, filed on Dec. 19, 2024.

(51) Int. Cl. A61B 17/00

(52) U.S. Cl. CPC A61B 17/00; A61B 2017/00805

(57) ABSTRACT

A non-invasive, externally worn sling-type device is disclosed for mitigating female urinary and fecal incontinence. The device comprises a hip-encircling belt and a vertical support strap assembly configured to apply controlled external pressure to the external urethral meatus and the anal orifice. The vertical support strap assembly includes an anterior strap portion, a central support member seamlessly integrated between the anterior and posterior strap portions, and a posterior strap portion adjustably coupled to the hip-encircling belt via a single G-hook anchor and flat attachment loop at the central rear of the hip-encircling belt. This configuration permits an initial, one-time customized adjustment of the length and tension of the vertical support strap assembly. All components are preferably formed of the same medical-grade thermoplastic elastomer (TPE) selected for hypoallergenic properties and durability. The device provides discreet mitigation of incontinence without internal insertion, surgical intervention, or absorbent pads, and is adaptable to varied anatomies and body types.

### CROSS-REFERENCE TO RELATED APPLICATIONS

This application claims the benefit of U.S. Provisional Application No. 63/736,263, titled “SLING-TYPE SUPPORT FOR PREVENTING FEMALE BLADDER AND BOWEL LEAKS”, filed Dec. 19, 2024, the entire contents of which are incorporated herein by reference in its entirety.

### FIELD OF THE INVENTION

The present invention relates to a non-invasive sling-type device for female continence support. More specifically, though not exclusively, the invention relates to a wearable device, comprising a hip-encircling belt and a vertical support strap assembly configured to span the perineum. This device is configured to apply controlled external pressure to the urethral and anal orifices, thereby mitigating episodes of urinary and fecal incontinence.

### BACKGROUND OF THE INVENTION

Urinary incontinence (UI) affects 25% to 60% of the global adult female population estimated at over two billion, with prevalence linked to age, childbirth trauma in younger women, and hormonal changes in older demographics. Fecal incontinence (FI) affects approximately 8% of adult women worldwide, while the co-occurrence of UI and FI, known as “dual incontinence,” is particularly common among older women and those in institutional settings.

In the United States alone, 61.8% of the more than 78 million adult women report experiencing some form of UI, based on weighted analyses of 2015-2018 National Health and Nutrition Examination Survey (NHANES) data, which confirm the global estimate. U.S. studies report similar prevalence for FI and dual incontinence, with rates also rising in older demographics and institutional settings. These widespread conditions across age groups underscore the need for a discreet, anatomically responsive, externally supportive device capable of addressing continence challenges throughout the female lifespan.

Existing prior art solutions for mitigating female urinary and fecal incontinence include absorbent products, mechanical supports, insertable aids, powered devices, and surgical implants. Conventional absorbent products manage leaked fluids rather than preventing them, and their bulk can lead to moisture buildup against the skin, increasing the risk of unpleasant odors, irritation, maceration, and infection. Other approaches intended to reduce or control symptoms often exhibit disadvantages such as invasiveness, discomfort, high cost, operational complexity, and the need for frequent maintenance, replacement, or adjustment.

Women experiencing urgency episodes often instinctively apply manual pressure to the external urethral meatus in response to involuntary bladder contractions, a long-recognized, instinctive attempt to relieve stress or urgency. The anal orifice, however, is not similarly accessible, leaving fecal incontinence unaddressed by such compensatory actions. Existing continence devices rely on internal supports, indwelling or implantable structures, or mechanically complex systems, and none provide a non-invasive, externally worn solution capable of delivering controlled pressure to both the external urethral meatus and anal orifice.

Accordingly, there remains a need for a discreet, anatomically responsive device that translates this instinctive manual behavior into a structured, wearable aid configured to apply controlled external pressure to both the external urethral meatus and the anal orifice. Such a device is preferably non-invasive and suitable for prolonged wear, while providing reliable external support without requiring internal insertion, surgical implantation, or powered components. In addition, the materials are preferably selected to permit irritation-free contact, thereby maintaining hygiene and discreet wear.

### SUMMARY OF THE INVENTION

The device of the present invention provides a discreet, anatomically responsive sling-type wearable aid intended for external use over conventional undergarments to mitigate urinary and fecal incontinence by applying controlled external pressure to the external urethral meatus and anal orifice. The device is preferably configured to accommodate anatomical variations across diverse body types and age groups.

The device includes a hip-encircling belt and a vertical support strap assembly, both preferably formed of a highly flexible and stretchable medical-grade thermoplastic elastomer (TPE). This construction allows the device to be easily donned and doffed by stretching it over the wearer's hips.

The hip-encircling belt is provided with an adjustable length mechanism to permit an initial, one-time adjustment of its circumference thereby ensuring a secure and customized fit.

In one or more embodiments, the vertical support strap assembly extends downward from the central front and rear of the hip-encircling belt and comprises an anterior strap portion, a central support member, and a posterior strap portion. The hip-encircling belt may be adjustable in circumference to accommodate variations in wearer anatomy and comfort. The anterior strap portion is slidably engaged with the hip-encircling belt to permit central positioning. The central support member is preferably configured to span the region between the external urethral meatus and anal orifice while extending slightly beyond these points at both extremities, thereby ensuring continuous coverage during movement and maintaining secure, comfortable support. The posterior strap portion is secured to the central rear of the hip-encircling belt and incorporates an adjustment mechanism engaged with a flat attachment loop via a single G-hook anchor to permit customizing the overall length and tension of the vertical support strap assembly to the wearer's anatomy.

In operation, the device functions by applying controlled external pressure to the external urethral meatus and anal orifice, thereby mitigating urinary and fecal incontinence without requiring internal insertion, muscular engagement, or reliance on pelvic floor activation. The materials used in the components of the device are selected for tensile strength, elasticity, low profile, and hypoallergenic properties, as well as biocompatibility and durability, thereby providing reliable continence support suitable for prolonged, daily use.

### BRIEF DESCRIPTION OF THE DRAWINGS

FIG. 1 illustrates an anterior view of the device, as worn on the lower abdomen of a female wearer, comprising a hip-encircling belt and the anterior portion of a vertical support strap assembly, according to one or more embodiments.

FIG. 2 illustrates a posterior view of the device, as worn on the lower back of a female wearer, comprising the hip-encircling belt and the posterior portion of the vertical support strap assembly, according to one or more embodiments.

FIG. 3 illustrates an inferior sectional schematic representation of a wearer's body at the level of the upper legs, according to one or more embodiments.

FIG. 4 is an enlarged detailed view of the posterior portion of the vertical support strap assembly illustrating the posterior strap portion with its upper extremity threaded through the closed end of a G-hook anchor, according to one or more embodiments.

FIG. 5 is an enlarged schematic representation of an embodiment of the single G-hook anchor utilized by the present device, according to one or more embodiments.

FIG. 6 is an enlarged cross-sectional view of the central support member, according to one or more embodiments.

FIG. 7 illustrates a profile view of the vertical support strap assembly, which includes tapered interfaces between the central support member and the anterior and posterior strap portions, respectively, as connected to the hip-encircling belt at both ends, according to one or more embodiments.

### DETAILED DESCRIPTION OF THE INVENTION

According to one or more embodiments, the present disclosure describes a wearable, non-invasive support device (100) and related methods for promoting continence. The underlying principle involves the application of controlled external pressure to the external urethral meatus (11) and the anal orifice (12), to reduce involuntary expulsion of urine, fecal matter, and intestinal gas. This pressure is delivered through an externally worn sling assembly, thereby eliminating the need for internal insertion, surgical implantation, or device-powered compression.

As shown in the accompanying figures, the support device (100) utilizes a streamlined design comprising a hip-encircling belt (13) and a vertical support strap assembly (14) extending downward from the central front and rear of the hip-encircling belt (13). According to one or more embodiments, the vertical support strap assembly (14) comprises three portions: an anterior strap portion (15), a central support member (17), and a posterior strap portion (16). The central support member (17) is physically distinct from the anterior and posterior portions in that it includes a semi-cylindrical section gradually tapered at both ends, which are integrated with the anterior strap portion (15) and posterior strap portion (16). This configuration provides a continuous structure connecting at its anterior extremity to the central front of the hip-encircling belt (13) and at its posterior extremity to the central rear of the hip-encircling belt (13).

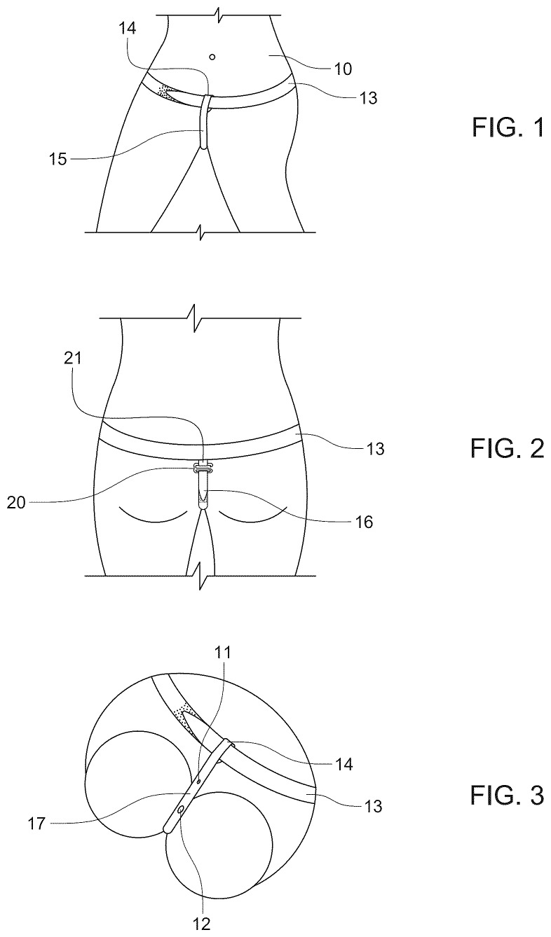

As shown in FIG. 1, an anterior view of an embodiment of the device (100) is depicted as worn on the lower abdomen (10) of a female wearer. The device (100) comprises the hip-encircling belt (13) and the anterior portion (15) of the vertical support strap assembly (14). The upper extremity of the anterior portion (15) forms a closed loop slidably engaged with the hip-encircling belt (13), while the lower extremity is integrated with the central support member (17). The anterior portion (15) may be of fixed length, with a preferred dimension of approximately 183 mm to 201 mm (7.2 in. to 7.9 in.), and preferably is not adjustable by the wearer, thereby serving as a stable reference against which the posterior portion (16) provides variability.

As shown in FIG. 2, a posterior view of an embodiment of the device (100) is depicted as worn on the lower back of a female wearer. The device (100) comprises the hip-encircling belt (13) and the posterior portion (16) of the vertical support strap assembly (14). The lower extremity of the posterior portion (16) is integrated with the central support member (17), and the upper extremity of the posterior portion (16) is adjustably engaged with the closed end of a G-hook anchor (20), the open end of which is engaged with a flat attachment loop (21) fastened beneath the hip-encircling belt (13), according to one or more embodiments.

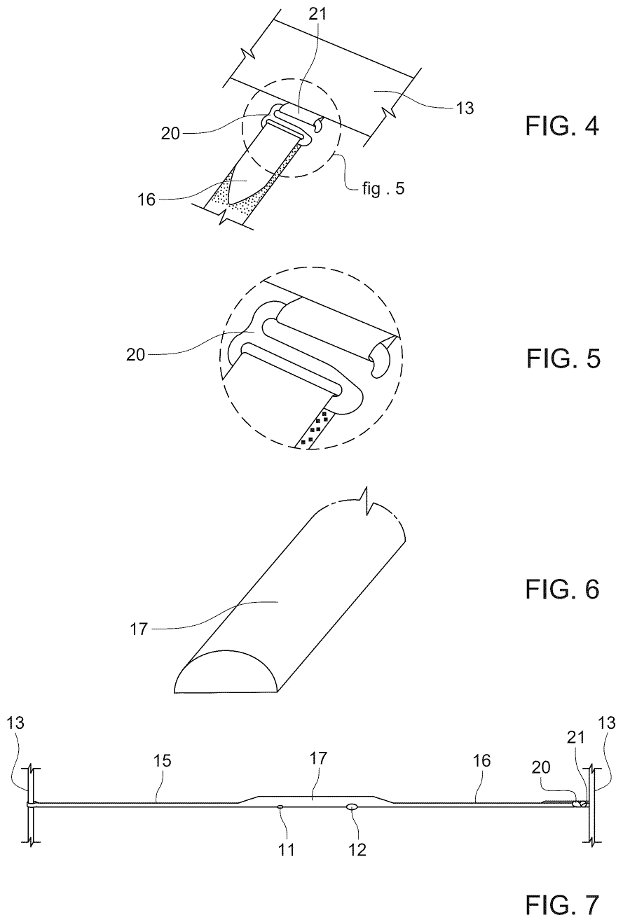

As shown in FIG. 4, an enlarged view of the upper extremity of the posterior portion (16) of the vertical support strap assembly (14) is depicted. The upper extremity of the posterior portion (16) is threaded through the closed end of the G-hook anchor (20). After threading, the upper extremity is folded downward and secured in place by a self-engaging fastener having both hook and loop components integrated on the upper surface of the posterior strap. The open end of the G-hook anchor (20) is engaged with the flat attachment loop (21) fastened beneath the hip-encircling belt (13). This configuration permits adjusting the length and tension of the vertical support strap assembly (14) as a whole so that it applies controlled pressure to the external urethral meatus (11) and the anal orifice (12), thereby mitigating female urinary and fecal incontinence, according to one or more embodiments.

FIG. 5 illustrates the single G-hook anchor (20) of FIG. 4.

As shown in FIG. 3, a schematic sectional representation in inferior view of the wearer's body at the level of the upper legs is depicted. It includes the abdomen (10), and the external urethral meatus (11) and the anal orifice (12). It further includes the hip-encircling belt (13), the anterior portion (15) of the vertical support strap assembly (14) looped around the hip-encircling belt (13) at its upper extremity, and the central support member (17) to which it is integrated at its lower extremity.

As shown in FIG. 6, an enlarged cross-sectional view of the central support member (17) is depicted, having an anatomically contoured, semi-cylindrical shape with a rounded surface configured for contact with the wearer's external urethral meatus (11) and anal orifice (12), and an opposing flat outer surface.

The preferred material used in the components of the support device (100), specifically medical-grade thermoplastic elastomer (TPE), is selected for its tensile strength, elasticity, low profile, and hypoallergenic properties, as well as its washability and durability for prolonged daily use. This material choice allows for a minimal-bulk design, providing an unobtrusive profile when worn under clothing.

According to one embodiment, the hip-encircling belt (13) is a single-component structure formed of a highly elastic and stretchable medical-grade thermoplastic elastomer (TPE). The belt has a width of approximately 15 mm to 20 mm (0.6 in. to 0.78 in.) and a variable length beginning at approximately 700 mm (28 in.). Its thickness is preferably approximately 1.0 mm to 2.0 mm (0.04 in. to 0.08 in.), providing a low-profile design while achieving a balance of support, flexibility, and durability.

The hip-encircling belt (13) is preferably worn below the anterior superior iliac spine (ASIS) for optimal positioning, comfort, and discretion. It is configured to be donned and removed by stretching it over the wearer's hips.

According to one or more embodiments, the circumference of the hip-encircling belt (13) is adjusted by the wearer upon first use to accommodate their anatomy. Adjustment is achieved by overlapping two sections of approximately 50 mm (2 in.) in length, disposed at both extremities of the belt, and comprising a self-engaging fastener with hook-and-loop components integrated on the same upper surface, such as VELCRO® Brand OMNI-TAPE® or ALFA-TEX®. This configuration allows for a one-time customized length and tension. A minimum overlap of approximately 25 mm (1 in.) is recommended to ensure secure attachment during use.

According to one embodiment, the vertical support strap assembly (14) comprises three portions: an anterior strap portion (15), a central support member (17), and a posterior strap portion (16). As shown in FIG. 7, a profile view of the vertical support strap assembly (14) illustrates the tapered interfaces of the central support member (17) with the anterior strap portion (15) and the posterior strap portion (16), as connected to the hip-encircling belt (13) at both ends.

According to one embodiment, the anterior strap portion (15) of the vertical support strap assembly (14) is preferably formed of the same medical-grade thermoplastic elastomer (TPE) as the other components of the device. The anterior strap portion (15) may have a width of approximately 8 mm to 10 mm (0.31 in. to 0.39 in.) and a length of approximately 183 mm to 201 mm (7.2 in. to 7.9 in.). Its thickness is preferably approximately the same as the hip-encircling belt (13), i.e., approximately 1.0 mm to 2.0 mm (0.04 in. to 0.08 in.). The upper extremity of the anterior strap portion (15) forms a closed loop that is slidably engaged with the hip-encircling belt (13), allowing for precise central positioning over the lower abdomen. The lower extremity of the anterior strap portion (15) is seamlessly integrated with the central support member (17).

The central support member (17) is preferably formed of the same medical-grade thermoplastic elastomer (TPE), selected for its soft texture, resilient elasticity, and non-slip finish. In one or more embodiments, the central support member (17) is configured with an anatomically contoured, semi-cylindrical cross-section. When worn and the overall length of the vertical support strap assembly (14) is properly adjusted, this configuration applies controlled external pressure to the external urethral meatus (11) and the anal orifice (12). The anterior and posterior extremities of the central support member (17) are seamlessly integrated with the anterior strap portion (15) and the posterior strap portion (16) of the vertical support strap assembly (14) via tapered interfaces.

In one embodiment, the central support member (17) has a length of approximately 40 mm to 55 mm (1.6 in. to 2.16 in.) and a semi-cylindrical cross-section with a diameter of approximately 8 mm to 10 mm (0.31 in. to 0.39 in.). This length corresponds to the anatomical distance between the external urethral meatus (11) and the anal orifice (12), typically 20 mm to 35 mm (0.8 in. to 1.4 in.), plus an extension of at least 10 mm (0.39 in.) beyond each landmark. In this way, the support member provides continuous coverage and effective support. The tapered interfaces where the central support member (17) integrates with the anterior strap portion (15) and the posterior strap portion (16) may transition over a length of approximately 10 mm (0.39 in.). The overall width of approximately 8 mm to 10 mm (0.31 in. to 0.39 in.) of the integrated components remains substantially constant across the two interfaces.

In one embodiment, the posterior strap portion (16) of the vertical support strap assembly (14) is preferably formed of the same medical-grade thermoplastic elastomer (TPE) as the other components of the device. The posterior strap portion (16) preferably has a width of approximately 8 mm to 10 mm (0.31 in. to 0.39 in.). Its thickness is preferably the same as the hip-encircling belt (13) and the anterior strap portion (15), i.e., approximately 1.0 mm to 2.0 mm (0.04 in. to 0.08 in.). The lower extremity of the posterior strap portion (16) is seamlessly integrated with the central support member (17), while the upper extremity is threaded through the closed end of a single G-hook anchor (20).

As shown in FIG. 4, the upper extremity of the posterior portion (16) of the vertical support strap assembly (14) is threaded into the closed end of the G-hook anchor (20), while the open end of the G-hook anchor (20) is hooked snugly into a flat attachment loop (21) formed of the same medical-grade thermoplastic elastomer (TPE). The G-hook anchor (20) preferably has a width of approximately 11 mm to 13 mm (0.43 in. to 0.51 in.) to accommodate the posterior strap portion (16) with sufficient tolerance. The flat attachment loop (21) is permanently fastened (e.g., bonded or overmolded) directly beneath the hip-encircling belt (13) at its center. In a preferred embodiment, the flat attachment loop (21) has a width of approximately 8 mm to 10 mm (0.31 in. to 0.39 in.), a thickness that is the same as the other components, i.e., approximately 1.0 mm to 2.0 mm (0.04 in. to 0.08 in.), and a length of approximately 8 mm to 12 mm (0.31 in. to 0.47 in.), the length being sufficient to allow the open end of the G-hook anchor (20) to pass through while fitting snugly so as to remain securely in place. The location of the G-hook anchor (20) on the lower back of the wearer maximizes comfort and stability during use, ensuring the rigid G-hook hardware does not contact sensitive anatomy.

As shown in FIG. 4, the upper extremity of the posterior portion (16), threaded into the closed end of the G-hook anchor (20) as previously described, is provided with a self-engaging fastener with both hook and loop components integrated on the same upper surface, such as a VELCRO® Brand OMNI-TAPER or ALFA-TEX®. This arrangement permits the upper extremity to be overlapped, thereby adjusting the length of the vertical support strap assembly (14). A minimum overlap of approximately 25 mm (1 in.) is recommended to ensure secure attachment during use. The anterior portion (15) preferably remains of fixed length.

Adjustment of the posterior strap portion (16) consequently determines the overall length of the vertical support strap assembly (14), preferably between approximately 400 mm and 450 mm (16 in. to 18 in.), together with its corresponding tension. This configuration permits the wearer to customize the effective length and tension of the vertical support strap assembly (14) to their anatomy, thereby ensuring that adequate pressure is applied to the external urethral meatus (11) and anal orifice (12) to mitigate episodes of urinary and fecal incontinence while maintaining wearer comfort. Such adjustment constitutes a important element in the full functionality of the device (100).

With respect to the use and function of the device (100), the coordinated configuration of the hip-encircling belt (13) and vertical support strap assembly (14) promotes ease of application by maintaining consistent anatomical alignment. Once initially adjusted, the hip-encircling belt (13) and vertical support strap assembly (14) retain their alignment and tension across repeated applications, including donning, doffing, and washing. The device (100) thereby preserves these settings to ensure consistent pressure delivery to the external urethral meatus (11) and the anal orifice (12) without requiring repeated adjustment over time, thereby enhancing both reliability and user convenience.

Furthermore, the highly elastic and stretchable medical-grade materials of the device (100) facilitate varied activities and enable independent use by individuals with limited range of motion or dexterity. The low-profile, hypoallergenic properties of these materials promote hygiene, support discreet wear, accommodate sensitive or aging skin, and maintain stability during extended periods of use. For toileting, wearers may either displace the vertical support strap assembly (14) together with their undergarments to the side for urination, particularly in environments where privacy, space, or hygiene are constrained, or lower both the device (100) and their undergarments for defecation, thereby preserving hygiene and maximizing convenience.

The hip-encircling belt (13) may be constructed in a plurality of basic sizes (e.g., small, medium, large) designed to approximate various anatomical measurements. In addition, each size may incorporate approximately 25 mm to 50 mm (1 in. to 2 in.) sections of self-engaging fastener at its free extremities, providing an adjustment range of approximately 25 mm to 38 mm (1 in. to 1.5 in.) through overlapping both extremities. This configuration further permits the wearer to connect multiple belt lengths end-to-end to achieve a customized fit, while minimizing excess material and maintaining a low profile for discreet wear.

The device (100) is configured to accommodate a wide range of anatomical variations, including postpartum changes, abdominal folds, and high body mass. The hip-encircling belt (13) and the vertical support strap assembly (14), which includes the central support member (17) configured to apply targeted pressure to the external urethral meatus (11) and the anal orifice (12), include smooth inner and outer surfaces, facilitating stable contact with abdominal folds and supporting ergonomic fit. In addition, the flexibility and adjustable length and tension of the components of the device (100) enable independent donning and doffing, thereby enhancing ease of use and accommodating anatomical diversity.

Furthermore, the medical-grade, hypoallergenic materials of the components of the device (100) permit it to be worn either over conventional undergarments or directly against the skin, according to the wearer's preference. In either configuration, the device (100) maintains safe contact and functional effectiveness for all body types without irritation.

Existing incontinence methods and devices have limitations and cannot guarantee complete cure or leakage prevention for every user. Accordingly, the device of the present invention (100) may be worn in conjunction with other incontinence products, including absorbent products, mechanical supports, insertable aids, or powered devices. When used in such combination, the device (100) provides a cooperative effect with enhanced protection and improved leakage control compared to use of conventional products alone, thereby offering increased confidence and security to the wearer in mitigating female urinary and fecal incontinence. The wearer should, however, confirm with a medical professional that the combination of devices does not create physical interference or contraindications for their specific medical condition.

In one or more embodiments, the hip-encircling belt (13) may be formed of textile webbing, thermoplastic polyurethane (TPU), or other flexible materials selected to balance comfort, durability, and manufacturing efficiency. The belt may be textured or finished to accommodate nesting or sliding mechanisms.

In one or more embodiments, the hip-encircling belt (13) and vertical support strap assembly (14) may incorporate low-profile adjustment mechanisms such as tri-glide sliders, ladder locks, cam buckles, or friction bars. These mechanisms are configured to resist slippage and maintain wearer-specified tension under daily wear conditions while minimizing bulk. Certain variants may include surface texturing, teeth, or other friction-enhancing features to ensure a secure hold. This dual adjustability accommodates anatomical variation and enables the wearer to modulate pressure.

In one or more embodiments, particularly those optimized for low-cost production or emergency use, the device (100) may be formed primarily of commercially available elastic bands of sufficient strength and width. In such variants, functionality relies on specific wearer positioning and optimized tensioning of the vertical support strap assembly (14). When formed of these commercially available materials, the wearer may use a thin panty liner within their conventional undergarments to provide additional hygiene and protection. Due to potential variations in material composition and hypoallergenic properties of commercial elastic bands, direct skin contact is not recommended in these specific embodiments unless the materials are explicitly confirmed to be hypoallergenic and clinically appropriate for the wearer's specific condition.

In one or more embodiments, the device (100) is configured for durability and reusability, thereby supporting broad accessibility across diverse resource settings. The device (100) may be particularly valuable in under-resourced regions and among low-income populations, providing a dignified, cost-effective alternative to conventional disposable products and invasive interventions.

In one or more embodiments, components of the device (100) may be formed of transparent, flesh-toned, or various colored options. Such embodiments promote wearer discretion, accommodate diverse cultural preferences, and potential requirements of clinical settings.

In one or more embodiments, the simplified geometry and compact, small-form design of the device (100) facilitate scalable, low-cost manufacturing. Suitable production methods include, but are not limited to, injection molding, overmolding, and extrusion processes.

The foregoing description provides illustrative and non-limiting examples of certain embodiments of the invention. Variations, modifications, and permutations that would be apparent to one of ordinary skill in the art are contemplated within the scope of the present disclosure. While the invention has been described with reference to specific exemplary embodiments, it will be understood that these embodiments are provided by way of example only, and not as limitations on the scope of the invention. The full scope of the invention is defined solely by the appended claims and their equivalents.

What is claimed is:

1. A wearable sling-type device for mitigating urinary and fecal incontinence in a wearer, the device comprising:
an adjustable hip-encircling belt configured to be worn around a wearer's lower abdomen;
a vertical support strap assembly extending downward from the central front and rear of the hip-encircling belt and comprising:
an anterior strap portion;
a central support member; and
a posterior strap portion;
wherein the upper extremity of the anterior strap portion forms a closed loop that is slidably engaged with the hip-encircling belt, and the lower extremity of the anterior strap portion is integrated with the central support member;
wherein the central support member is dimensioned to span between the wearer's external urethral meatus and anal orifice and is configured to apply controlled external pressure to said landmarks; and
wherein the lower extremity of the posterior strap portion is integrated with the central support member, and the upper extremity of the posterior strap portion is adjustably coupled to the hip-encircling belt via a single G-hook anchor engaged with a flat attachment loop at the central rear of the hip-encircling belt.

2. The device of claim 1, wherein the device is formed of a highly elastic and stretchable medical-grade thermoplastic elastomer.

3. The device of claim 2, wherein the medical-grade thermoplastic elastomer is selected for its tensile strength, low profile, hypoallergenic properties, biocompatibility, washability, and durability for prolonged daily use.

4. The device of claim 1, wherein the hip-encircling belt has a width of approximately 15 mm to 20 mm and a thickness of approximately 1.0 mm to 2.0 mm.

5. The device of claim 4, wherein the hip-encircling belt has an adjustable length starting at approximately 700 mm and comprises opposing ends with a self-engaging fastener at each opposing end to enable adjustable circumference using an overlap section of approximately 50 mm.

6. The device of claim 1, wherein the vertical support strap assembly has an approximate overall adjustable length of 406 mm to 457 mm.

7. The device of claim 1, wherein the anterior strap portion of the vertical support strap assembly has a width of approximately 8 mm to 10 mm, a thickness of approximately 1.0 mm to 2.0 mm, and a length of approximately 183 mm to 201 mm.

8. The device of claim 1, wherein the central support member is configured with an anatomically contoured, semi-cylindrical cross-section.

9. The device of claim 8, wherein the central support member has a diameter of approximately 8 mm to 10 mm, and a length of approximately 40 mm to 55 mm.

10. The device of claim 9, wherein the length of the central support member includes approximately 10 mm extensions beyond the external urethral meatus and anal orifice of a wearer's perineum to accommodate an anatomical length of approximately 20 mm to 35 mm.

11. The device of claim 9, wherein the central support member integrates with the anterior strap portion and the posterior strap portion via a tapered interface that transitions over a length of approximately 10 mm, and wherein the overall width of the integrated components remains substantially constant across the two interfaces.

12. The device of claim 1, wherein the posterior strap portion has a width of approximately 8 mm to 10 mm, a thickness of approximately 1.0 mm to 2.0 mm, and a length of approximately 183 mm to 201 mm.

13. The device of claim 11, wherein the posterior strap portion is configured for tension adjustment using a self-engaging hook-and-loop fastener with an overlap section of approximately 100 mm.

14. The device of claim 1, wherein the single G-hook anchor is secured to the flat attachment loop and is positioned on the lower back of the wearer to maximize comfort and stability during use, ensuring the rigid single G-hook hardware does not contact sensitive anatomy.

15. The device of claim 1, wherein the flat attachment loop has a width of approximately 8 mm to 10 mm, a thickness of approximately 1.0 mm to 2.0 mm, and a length of approximately 8 mm to 12 mm.

16. The device of claim 1, wherein the single G-hook anchor has a width of approximately 11 mm to 13 mm to accommodate the posterior strap portion with sufficient tolerance to avoid breakage.

17. A method for mitigating urinary and fecal incontinence in a female wearer using the wearable sling-type device of claim 1, the method comprising the steps of:
engaging the anterior strap portion as a closed loop slidably with the hip-encircling belt;
adjustably coupling the upper extremity of the posterior strap portion to the hip-encircling belt via a single G-hook anchor and a flat attachment loop; and
applying a controlled external pressure to the wearer's external urethral meatus and anal orifice via the central support member by adjusting the tension of the vertical support strap assembly.

18. The method of claim 17, wherein all components of the device are formed of a medical-grade thermoplastic elastomer (TPE), and the step of adjustably coupling is performed using a self-engaging hook-and-loop fastener that provides a one-time customized adjustment.

19. The method of claim 17, further comprising the step of positioning the single G-hook anchor on the lower back of the wearer to ensure rigid hardware does not contact sensitive anatomy during physical activity.
